(19) United States
(12) Patent Application Publication
(10) Pub. No.: US 2004/0259131 A1
(43) Pub. Date: Dec. 23, 2004

(54) Methods for detection of target molecules and molecular interactions

(75) Inventors: Stuart Wilson, London (GB); Christopher John Stanley, Cambridge (GB)

Correspondence Address: GREENBERG TRAURIG, LLP, ONE INTERNATIONAL PLACE, 20th FL, ATTN: PATENT ADMINISTRATOR, BOSTON, MA, 02110, US

(73) Assignee: Iseao Technologies Limited

(21) Appl. No.: 10/817,225

(22) Filed: Apr. 2, 2004

Related U.S. Application Data
(63) Continuation of application No. PCT/GB02/04489, filed on Oct. 3, 2002.

(30) Foreign Application Priority Data
Oct. 3, 2001 (GB) 0123754.4
Oct. 10, 2001 (GB) 0124330.2
May 30, 2002 (GB) 0212544.1

(51) Int. Cl. C12Q 1/68

(52) U.S. Cl. 435/6

(57) ABSTRACT

The invention relates to methods of detecting target molecules or the interactions between target molecules based on interactions between proximally-bound nucleic acid tags. Reagent kits for use in such methods are also provided.

### FIELD OF THE INVENTION

The invention relates to methods and kits for detecting target molecules or the interactions between target molecules.

### BACKGROUND TO THE INVENTION

Sensitive methods exist to detect target molecules such as particular nucleic acids, proteins or more simple molecules. The presence of such molecules may be used to indicate an on-going infection or environmental contamination, for example. In order for these methods to be very sensitive and to detect as little as a single molecule the methods must also have high specificity. This high specificity is often achieved by binding two reporters to the target molecule that is to be detected.

In the case of the highly sensitive polymerase chain reaction (PCR), for example, two short nucleic acid probes or primers recognise the target nucleic acid. The detection of the target nucleic acid is thus only achieved when both primers are bound to, and linked through, the same target molecule. Non-specific interactions of the primers with other molecules are not detected unless both primers bind to and are linked by this non-specific interaction. The conditions of the reaction are such that the latter is highly unlikely. This PCR method and other molecular amplification methods, well known in the art, such as NASBA (Compton, 1991) and 3SR (Fahy et al., 1991) can be used to detect target nucleic acids.

In order to detect other molecules such as proteins an antibody specific for the target molecule can be linked to a nucleic acid sequence that is subsequently detected. In this example, however, the high specificity of detection is lost as there is only one target-binding antibody.

Target molecules can, however, be detected in a very sensitive and specific manner through the Dual Phage approach. In the Dual Phage assay two phage are needed in order to generate a signal and the two phage must be brought together or linked through the target molecule. This is most easily achieved by linking the phage to ligands such as antibodies that are specific for the target molecule. In this method it is possible to detect a wide range of target molecules including nucleic acids, proteins and simple or complex molecules.

In another approach disclosed in US Patent Nos. U.S. Pat. No. 5,635,602 and U.S. Pat. No. 5,665,539 two target-specific antibodies are both linked to the same piece of nucleic acid, such that the nucleic acid forms a bridge. After binding to the target this nucleic acid bridge is specifically cleaved and then re-associated. The presence of an intact nucleic acid bridge (i.e. cleaved and re-associated) is shown by the use of PCR and two primers that recognise the reformed nucleic acid, because the nucleic acid bridge contains two PCR primer binding sites. This approach enhances the specificity of the assay because the nucleic acid is more likely to reform after cleavage if both antibody molecules are bound to the target and are thus in close spatial proximity. Reforming of the nucleic acid bridge is unlikely to happen if only one antibody is bound non-specifically to a molecule other than the intended target. A disadvantage of this approach is the problem of ensuring that all of the nucleic acid bridge molecules are cleaved in the absence of target antigen. In addition, the method is complex and involves a number of steps that could involve DNA restriction enzymes, DNA polymerase and DNA ligation enzymes.

EP 0 832 431 relates to processes for immunological detection of a specific antigen. The method is based on use of a first immobilized reagent having affinity to a specific macromolecule, and second and third affinity reagents specific for different determinants of the macromolecule. The second and third reagents are modified with oligonucleotides that can be cross-linked when the reagents are held in close proximity, allowing amplification of the cross-linked oligonucleotides, and thus detection of the specific antigen.

WO 01/61037 relates to assays for detection of analytes in solution using so-called proximity probes. Said proximity probes consist of a binding moiety and a nucleic acid. Upon binding of the proximity probes to the analyte the nucleic acids are brought into close proximity and can thus be ligated and then detected, usually by amplification. This technique has the disadvantage that ligation can be an inefficient reaction. Also the ligase must be removed from the reaction mixture before the detection process can be carried out.

Sensitive methods are also needed for monitoring molecular interactions. Drug discovery and proteomics rely on the monitoring of such interactions. A variety of methods are currently in use, which are well known in the art, such as the Scintillation Proximity Assay (SPA) (Bosworth and Towers, 1989) and various Yeast Hybrid methodologies (Ma and Ptashne, 1988; Fields and Sternglanz, 1994).

The Dual Phage method can also be applied to the monitoring of molecular interactions. In this case, the molecules whose interaction is to be studied each have a ligand-binding site that can bind one phage type either directly or indirectly. The interaction of the molecules is thus able to be monitored through the linking of the two phage types. If the molecules interact the two phage types are brought together but if they do not interact the two phage types remain separate. This approach can be applied to proteomics and drug discovery.

### DESCRIPTION OF THE INVENTION

The present invention seeks to provide improved methods for detecting the binding of two or more binding entities to a target molecule and for monitoring molecular interactions.

According to a first aspect of the invention, referred to hereafter as the “detection method”, there is provided a method of detecting a target molecule comprising the steps of:

a) contacting a sample with two or more binding entities;

b) allowing the binding entities to bind to the target molecule;

c) allowing interaction between nucleic acid tags attached to the binding entities, wherein the interaction generates at least one tag comprising novel sequence, and wherein the nucleic acid tags are not covalently cross-linked following the interaction;

d) detection of novel sequence in at least one tag generated in step c).

The method relies on interaction between nucleic acid tags attached to binding entities specific for the target molecule of interest, this interaction generating at least one tag having novel sequence. In this context “novel sequence” means a sequence which is different to the sequences of the nucleic acid tags present before the tags were allowed to interact. The generation of novel sequence occurs by a process of interaction between nucleic acid tags brought into close proximity via binding of the binding entities to proximal binding sites on the target molecule.

Binding of the binding entities to the target molecule has the effect of bringing the nucleic acid tags into close proximity, thus facilitating interaction between them. The close juxtaposition of the nucleic acid tags resulting from binding of binding entities to proximal sites on the target enhances the specificity of the detection reaction and also results in a rate enhancement that provides an improved signal to noise ratio over the background due to non-specific binding that may result in random sequence generation events.

In the most preferred embodiment of the detection method the nucleic acid tags are not covalently cross-linked following the interaction. This means that nucleic acid tags attached to separate binding entities (or interacting molecules) prior to the interaction do not become permanently linked as a result of the interaction, regardless of whether or not any “transient” cross-liking occurs during the interaction. As a result of the lack of permanent cross-linking no nucleic acid “bridging” structure is formed between the binding entities/interacting molecules. Typically the interaction generates separate tags, at least one of which has a novel sequence. Most preferably, the method involves interaction between two nucleic acid tags to generate two separate tags, each having novel sequence. In this latter embodiment there is effectively an “exchange” of nucleic acid between the two tags as a result of the interaction.

The fact that the nucleic acid tags are not covalently linked following the interaction can provide significant technical advantages with regard to detection of novel sequence tags generated by the interaction. For example, generation of two separate tags, each having novel sequence, provides two independently verifiable products. Furthermore, if detection of the novel sequence is to be accomplished by an amplification reaction, this reaction may be more efficient if free of potential steric constraints that might be present if the tags were to remain physically cross-linked during the amplification step.

The nucleic acid tags may “interact” by any type of reaction which leads to the generation of at least one tag of novel sequence, wherein the tags are not covalently linked. In the most preferred embodiment of the invention, interaction between the nucleic acid tags occurs by recombination.

The use of recombination as a means of interaction between proximal nucleic acid tags of itself provides technical advantages over prior art methods, for example ligation, regardless of whether or not the nucleic acid tags become cross-linked as a result of the interaction.

Therefore, the invention also provides a method of detecting a target molecule comprising the steps of:

a) contacting a sample with two or more binding entities;

b) allowing the binding entities to bind to the target molecule;

c) allowing recombination between nucleic acid tags attached to the binding entities thus generating novel sequence;

d) detection of novel sequence generated by recombination between the nucleic acid tags.

“Recombination” is defined herein to include any exchange of nucleic acid sequence or deletion or insertion of sequences between the nucleic acid tags in order to generate at least one novel sequence that is capable of being detected. Examples include site-specific recombination events (e.g. requiring a specific recombinase), transposition events (e.g. requiring a specific transposase), and homologous recombination events.

Site-specific recombination events are non-homologous recombination events, in so far as they generally do not require extensive homology between the nucleic acid sequences. In most cases site-specific recombination requires the presence of short recombination site sequences (generally a few tens of basepairs). Many site-specific recombination systems require the presence of identical recombination site sequences on the interacting nucleic acid molecules. However, in other systems the recombination sites may share little or no sequence homology, as is the case with the integration sites attP and attB, derived respectively from bacteriophage lambda and the E. coli chromosome.

In a preferred embodiment recombination between nucleic acid tags, leading to generation of novel sequence, is catalysed by a recombinase. This may be achieved by inclusion of site-specific sequences in the nucleic acid tags that are recognised by a specific recombinase enzyme.

Suitable site-specific recombination systems which may be used include the Cre/loxP system, wherein the nucleic acid tags contain loxP sites and recombination catalysed by Cre recombinase. Another suitable system is the bacteriophage lambda integration system, wherein the nucleic acid tags contain attP and attB recognition sequences or attL and attR sequences, allowing recombination catalysed by an enzyme which recognises these sites. Recombination between attB and attp sites or between attL and attR sequences is catalysed by the lambda phage enzyme integrase, and requires a host-accessory factor IHF. The lambda phage recombination system is well known in the art and the enzymes required for recombination are available commercially (e.g. as components of the Gateway™ cloning system supplied by Invitrogen). These particular recombination systems are listed by way of example only and it is not intended to, limit the invention to the use of these specific systems. Other site-specific recombination systems known in the art such as, for example, the Flp/FRT system may also be used.

In a still further embodiment, recombination may depend upon a transposition event and rely upon the use of a transposase.

One suitable example of such a recombination system is one which depends upon Tn5 transposase that recognizes Mosaic Ends recognition sequences. However, it is not intended to limit the invention to the use of this specific system, and other transposition systems known in the art may be used.

In a further embodiment one nucleic acid tag bound to one binding entity contains a transposable element recognized by a transposase that can transpose into a nucleic acid tag bound to a second binding entity. In this example it is not necessary for all nucleic acid tags to contain a site-specific recombination or transposition site.

In a further embodiment recombination may occur by homologous recombination. If detection of the novel sequence generated by homologous recombination is to be carried out by an amplification reaction, then it is advantageous to position the primer binding sites required for the amplification at the extreme ends of the nucleic acid tags, and outside of the region of homology, such that any recombination event occuring anywhere between the primer binding sites can be detected by amplification using the novel combination of primer-binding sites.

In order to achieve site-specific recombination, nucleic acid tags bearing the correct recombination site sequences must be brought into close proximity in the presence of the appropriate recombinase (or transposase). The recombinase or transposase enzyme may be present in the reaction medium in which the binding steps of the method are carried out, or the enzyme may be added in a separate reagent addition step, for example following binding of the binding entities to the target molecule.

In certain embodiments it may be advantageous to start with the enzyme in an inactivated state and then activate the enzyme only after binding of the binding entities to the target molecule has taken place to position the nucleic acid tags in close proximity. Activation of the enzyme may, for example, be achieved by changing the composition, pH or temperature of the reaction medium.

In the context of this invention “binding entities” are defined as any molecule that can bind specifically to a target molecule. Binding entities include, for example, antibodies, lectins, receptors, transcription factors, cofactors and nucleic acids, and fragments thereof which retain target-specific binding activity (e.g. Fab fragments). This list is merely illustrative and is not intended to be limiting to the invention.

The target molecule itself can be any molecule for which it is desired to provide a specific detection method. The target molecule may comprise a single molecule, a multimer, aggregate or molecular collection or complex. A multimer will generally comprise a number of repeats of a single molecule linked together through covalent or non-covalent interactions. A complex will generally consist of different molecules interacting through covalent or non-covalent interactions.

The binding entities may bind different regions of a single target molecule. Thus, the nucleic acid tags will be brought into close proximity when the binding entities bind to their respective regions of the target molecule.

If the target molecule is a multimer or aggregate, then the binding entities may bind to equivalent binding sites on the monomeric components of the multimer or units making up the aggregate.

Suitable methods of linking the nucleic acid tags to binding entities are known in the art, see for example the techniques described in manuals such as Bioconjugation; M Aslam and A Dent, eds. Macmillan Reference Ltd 1998.

In a preferred embodiment the binding entities may be labeled with multiple copies of the nucleic acid tags which will enhance the sensitivity of detection. Most preferably there will be between about 1 and 100 copies of nucleic acid tag per copy of binding entity, although a greater number is also within the scope of the present invention.

In a preferred embodiment the nucleic acid tags may be attached directly to the binding entities. Direct linkage may be achieved via a covalent linkage.

Amine derivatized nucleic acid tags may be coupled to the binding entities using any one of a number of chemical cross-linking compounds.

It is also within the scope of the invention to have the nucleic acid tags attached indirectly to the binding entities. For example the attachment may be achieved through linker molecules. Suitable linker molecules include components of biological binding pairs which bind with high affinity, for example biotin/streptavidin or biotin/avidin.

For most applications of the detection method the tags will be attached to the binding entities at the start of the detection reaction, at least before the binding of the binding entities to the target molecule. Most preferably, the binding entities will be supplied pre-labelled with nucleic acid tags, or else the tags will be attached in a separate reagent labelling step. However, the possibility of attaching the nucleic acid tags to the binding entities during the detection reaction itelf, i.e. following binding of the binding entities to the target, is not excluded.

“Nucleic acid tags” are defined herein to include any natural nucleic acid and natural or synthetic analogues that are capable of interaction to generate novel sequence, for example by recombination. Suitable nucleic acid tags include tags composed of double or single-stranded DNA, double or single-stranded RNA. Tags which are partially double-stranded and partially single-stranded are also contemplated. It is also comtemplated to use single-stranded tags in combination with double-stranded tags, i.e. one component labelled with a single-stranded tag and another component labelled with a double-stranded tag capable of interacting with the single-stranded tag. If the interaction is to be achieved by recombination then the nucleic acid tags may be composed of any nucleic acid which is capable of participating in a recombination reaction, suitable examples including linear or circular double-stranded DNA (dsDNA) or double-stranded RNA (dsRNA) or mixtures thereof. Most preferably the nucleic acid tags will comprise dsDNA. The term “nucleic acid” encompasses includes synthetic analogues which are capable of “interacting” in an analogous manner to natural nucleic acids, for example nucleic acid analogues incorporating non-natural or derivatized bases, or nucleic acid analogues having a modified backbone. In particular, the term “double-stranded DNA” or “dsDNA” is to be interpreted as encompassing dsDNA containing non-natural bases.

The precise sequence of the nucleic acid tags is not material to the invention, except to the extent that certain sequences may be required to enable the “interaction” between the tags, thus generating at least one tag of novel sequence. For example, specific sequences are required to permit site-specific recombination. The tags attached to the different binding entities will most usually be of different sequence, so that an interaction event between the nucleic acid tags leads to production of at least one novel sequence that can be detected. However, it is not excluded to use tags of identical sequence, provided that the tags are able to interact to generate novel sequence.

The step of detecting the novel sequence generated by interaction between the nucleic acid tags may be accomplished using any suitable technique known in the art.

Most preferably, detection of the novel sequence will involve an amplification reaction, for example PCR, NASBA, 3SR or any other amplification technique known in the art. Amplification is achieved with the use of amplification primers specific for the novel sequence. In order to provide specificity for the novel tag sequence primer binding sites corresponding to a region of completely novel sequence may be selected, or else a novel combination of primer binding sites, not present in the original tags, may be chosen.

The skilled reader will appreciate that the novel sequence may also include sequences other than primer binding sites which are required for detection of the novel sequence, for example RNA Polymerase binding sites or promoter sequences required for isothermal amplification technologies, such as NASBA or 3SR.

In a preferred embodiment detection of the novel sequence is carried out by amplification with “real-time” detection of the products of the amplification reaction. This can be achieved using any amplification technique which allows for continuous monitoring of the formation of the amplification product.

A number of techniques for real-time detection of the products of an amplification reaction are known in the art. Many of these produce a fluorescent read-out that can be continuously monitored, specific examples being molecular beacons and fluorescent resonance energy transfer probes. Real-time quantification of PCR reactions can be accomplished using the TaqMan® system (Applied Biosystems).

In a most preferred embodiment the entire detection method is carried out in real-time, meaning that binding of the binding entities, interaction of the nucleic acid tags and detection of the product of the interaction are carried out simultaneously in a single reaction step. Real-time detection requires that the binding step, interaction between the nucleic acid tags, and detection of the product of the interaction can all be carried out under a single set of reaction conditions, without the need for intermediate washing steps. This can be achieved if the interaction between the nucleic acid tags is carried out by recombination, and represents a major technical advantage of the present invention over the prior art. In this embodiment real-time detection of the novel sequence will preferably be carried out using an isothermal amplification reaction, for example NASBA or 3SR, in order to avoid changes of temperature which might adversely affect the binding of the binding entities to the target molecule.

In a further preferred embodiment the method of the invention may be-performed in the presence of one or more competitor molecules. This embodiment will most preferably be carried out entirely in solution. The competitor molecules will preferably be present in excess. The competitor molecules are designed so that they can interact with at least one of the nucleic acid tags. However, they have a different sequence such that the interaction cannot lead to generation of novel detectable sequence on the nucleic acid tags. In one preferred approach the competitor-can exchange sequence with the nucleic acid tags by recombination. Once the competitor has interacted with at least one of the nucleic acid tags, the tags can no longer interact to generate novel sequence. In the absence of binding of the binding entities to target molecules, excess nucleic acid tags in free solution are “mopped up” or inactivated before they can interact and generate detectable novel sequence. In the presence of binding of the binding entities to target molecules, the spatial proximity of the nucleic acid tags allows the tags to interact with each other to generate detectable novel sequence, rather than interacting with the competitor molecules.

Use of competitor molecules enables the method to be carried out entirely in solution without having to immobilise any of the target molecules. Use of competitor molecules also decreases the background signal due to tags interacting in the absence of specific binding of binding entities to the target molecule.

The present invention provides an improvement over prior art methods, since sensitive and specific detection of a target molecule can be acheived in a single reaction step without the need for intermediate washing steps. Thus the method is more amenable to automation, for example in a high throughput context. Also recombination, as catalysed by recombinases and transposases, is generally a more efficient method than ligation, meaning that the sensitivity and reproducibility of the method will be improved compared to methods which rely on the use of ligation.

The “detection method” of the invention may be adapted for the detection of essentially any “target molecule” for which suitable “binding entities” of the required specificity are available. The “sample” to be tested using the method may be essentially any material which permits the specific binding reactions that are essential to the operation of the detection method.

The “detection method” is of use in all areas of technology where it is desirable to provide specific detection of target molecules, in particular target biological molecules such as proteins, nucleic acids, carbohydrates, etc. One important area of application of the detection method, though not intended to be limiting, is in the field of clinical diagnostics. Typically the “sample” will be a sample of biological fluid, e.g. whole blood, serum, plasma, urine etc, taken from a human patient. Other important applications may include the field of environmental testing and monitoring.

In an important embodiment of the invention, the features of the detection method described above may be adapted in order to monitor molecular interactions.

Therefore, in accordance with a second aspect of the invention, referred to herein as the “interaction method” there is provided a method of detecting interactions between two or more interacting molecules comprising the steps of:

a) incubating the interacting molecules such that they can interact;

b) allowing interaction between nucleic acid tags attached to the interacting molecules, wherein the interaction generates at least one tag comprising novel sequence, and wherein the nucleic acid tags are not covalently cross-linked following the interaction;

c) detection of novel sequence on at least one tag generated in step b).

The invention further provides a method of detecting a target molecule comprising the steps of:

a) contacting a sample with two or more binding entities;

b) allowing the binding entities to bind to the target molecule;

c) recombination between nucleic acid tags attached to the binding entities thus generating novel sequence;

d) detection of novel sequence generated by recombination between the nucleic acid tags.

The “interaction method” may be used in essentially any field of technology where it is desired to monitor interactions between molecules, and particularly interactions between biological molecules.

In a preferred embodiment, the interaction method may be used in proteomics in order to investigate molecular interactions. For example a first interacting molecule may be labelled with a first nucleic acid tag, and a library of molecules which may potentially interact with the first interacting molecule may then each be labeled with a second nucleic acid tag. If an interaction occurs between the first interacting molecule and a component from the library of molecules, this brings the first and second tags into close proximity, thus allowing interaction between the tags, to generate a novel sequence which can be detected in order to identify interacting partners.

A further application is in the field of drug discovery. For example, the interaction method may be used to study interactions between particular combinations of molecules and to identify potential inhibitors or enhancers of molecular interactions. Potential inhibitors of a given interaction could be identified by screening for the ability to reduce the signal detected following interaction of nucleic acid tags brought into close proximity by interaction between the interacting molecules.

The “interacting molecules” may be essentially any combination of interacting molecules which it is desired to study. These may be, for example, subunits of a multi-subunit complex, a pair of monomers making up a dimer, a ligand and receptor, an enzyme and substrate or inhibitor, etc.

The “interaction method” differs from the detection method only in that the nucleic acid tags are attached to the interacting molecules which it is desired to evaluate, rather than to binding entities capable of binding to a target molecule. The interaction method may therefore incorporate analogous features to those described above in connection with the detection method, as would be apparent to the skilled reader.

It is particularly preferred to carry out the interaction method in real-time, using the approaches described above in connection with the detection method. The ability to monitor molecular interactions in real-time provides significant advantages, particularly in the field of drug discovery.

The invention also relates to reagent kits suitable for use in carrying out the detection method or the interaction method.

Reagent kits suitable for use in carrying out the detection method may comprise two or more binding entities each labelled with nucleic acid tags, characterized in that the nucleic acid tags are capable of interacting to generate at least one tag comprising novel sequence, wherein the nucleic acid tags are not covalently cross-linked following the interaction.

In a preferred embodiment the nucleic acid tags are capable of interacting by recombination. Therefore, the invention further provides a reagent kit comprising two or more binding entities each labelled with nucleic acid tags, characterized in that the nucleic acid tags are capable of recombination to generate at least one tag having novel sequence.

Reagent kits for use in carrying out the interaction method may comprise two or more interacting molecules each labelled with nucleic acid tags, characterized in that the nucleic acid tags are capable of interacting to generate at least one tag comprising novel sequence, wherein the nucleic acid tags are not covalently cross-linked following the interaction.

In a preferred embodiment the nucleic acid tags are capable of interacting by recombination. Therefore, the invention further provides a reagent kit comprising two or more interacting molecules each labelled with nucleic acid tags, characterized in that the nucleic acid tags are capable of recombination to generate at least one tag having novel sequence.

The reagent kits may incorporate any of the preferred features mentioned in connection with the detection and interaction methods. For example, the nucleic acid tags included in the kits may incorporate recombination sites which allow interaction via site-specific recombination, catalysed by a recombinase or a transposase. Preferred combinations of recombination sites and enzymes are as listed above in the description of the detection and interaction methods. The kits may further comprise a supply of a suitable recombinase or transposase, and may include supplies of any enzyme co-factors, accessory proteins etc which are required for the recombination reaction.

Reagent kits may further include supplies of suitable reaction buffer(s). Where detection of the product of the interaction between the nucleic acid tags is to be achieved by amplification, the kit may also include reagents required for the amplification reaction, for example the kit may include any of the following: primer sets, amplification enzymes, probes for detection of the amplification product (including probes labelled with fluorescent or other revealing labels), positive control amplification templates, reaction buffers etc.

The invention still further relates to a reagent labelling kit which may be used to label interacting molecules or binding entities for use in the interaction or detection methods, the kit comprising two or more nucleic acid tags and means for attaching the tags to interacting molecules/binding entities, characterized in that the kits contain at least one pair of nucleic acid tags which are capable of interacting to generate at least one nucleic acid tag having a novel sequence, wherein the two tags are not covalently cross-linked following the interaction.

Again, the nucleic acid tags are most preferably capable of interacting by recombination. Therefore, in a preferred embodiment the reagent-labelling kit may comprise two or more nucleic acid tags and means for attaching the tags to interacting molecules, characterized in that the kit contains at least one pair of nucleic acid tags which are capable of recombination to generate at least one tag having novel sequence.

In one embodiment the means for attaching the tags to interacting molecules or binding entities may be a chemical reagent capable of cross-linking nucleic acid to a binding entity or interacting molecule.

In a preferred embodiment the “means for attaching the tags” may be an indirect linkage. Preferred types of indirect linkage are provided by components of a biological binding pair, for example biotin/avidin or biotin/streptavidin. In this embodiment at least one of the nucleic acid tags is conjugated with one half of the biological binding pair. The kit may contain a supply of pre-conjugated nucleic acid tags, or may include tags which have not yet been conjugated together with means for conjugating the tags with half of the binding pair.

The kit will further include either binding entity or interacting molecule pre-conjugated with the other half of the binding pair, or else means for conjugating a binding entity or interacting molecule of choice to the other half of the binding pair.

The means for attaching half of the biological binding pair to an interacting molecule may (depending on the nature of the binding pair) be a chemical cross-linking reagent. However, it may comprise an expression vector which can be used to express the binding entity or interacting protein as a fusion protein, either as a direct fusion with the other half of the binding pair or as a fusion with a polypeptide tag which enables attachment of the other half of the binding pair. By way of example, vectors for the expression of biotinylated fusion proteins are known in the art and are commercially available (for example the PinPoint vector system from Promega, Madison, Wis., USA). These vectors allow expression of proteins as fusions with a biotinylation domain of the biotin carboxylase carrier protein. The fusion proteins can be biotinylated in E. coli host cells in an ATP-dependent enzymic reaction. Thus, the reagent labelling kit may contain a supply of such a vector, which enables expression of biotinylated fusion proteins, plus streptavidin conjugated nucleic acid tags.

The invention will be understood with reference to the following examples, together with the accompanying drawings in which:

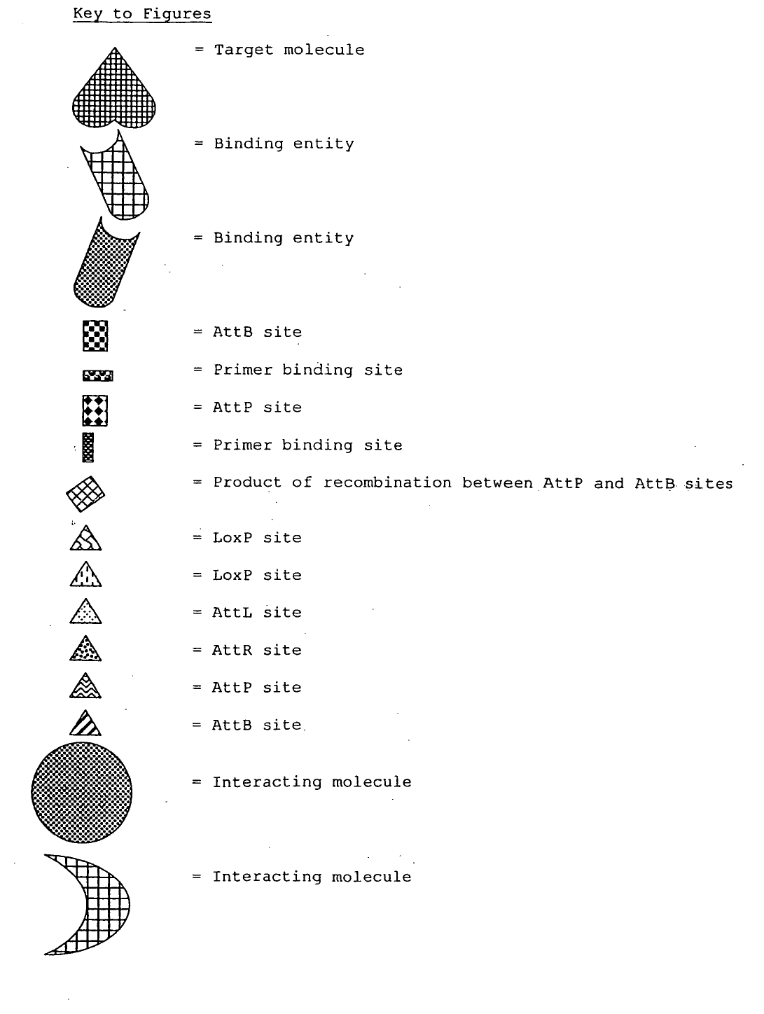

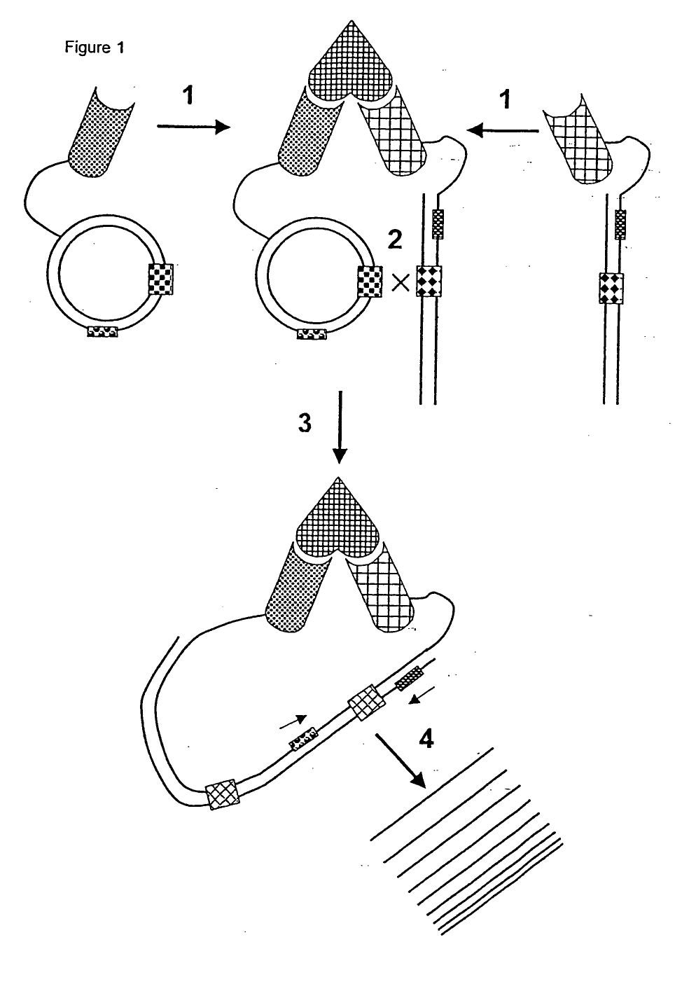

FIG. 1 schematically illustrates an embodiment of the detection method using two binding entities labelled, respectively, with circular and linear double-stranded DNA tags.

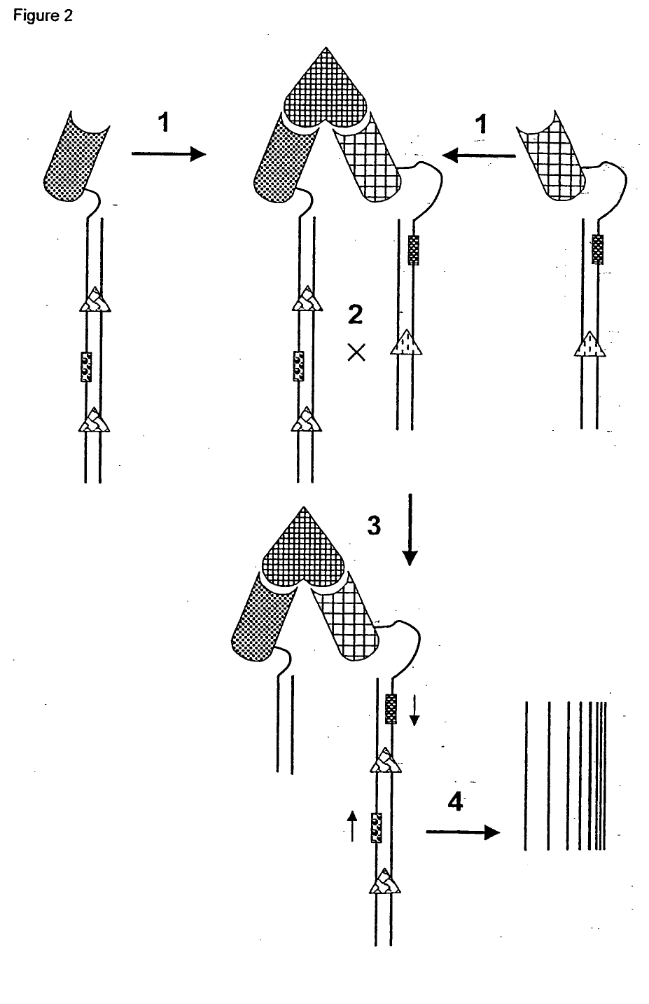

FIGS. 2 to 4 schematically illustrate embodiments of the detection method using two binding entities labelled with linear double-stranded DNA tags, containing different arrangements of site-specific 10 recombination sites.

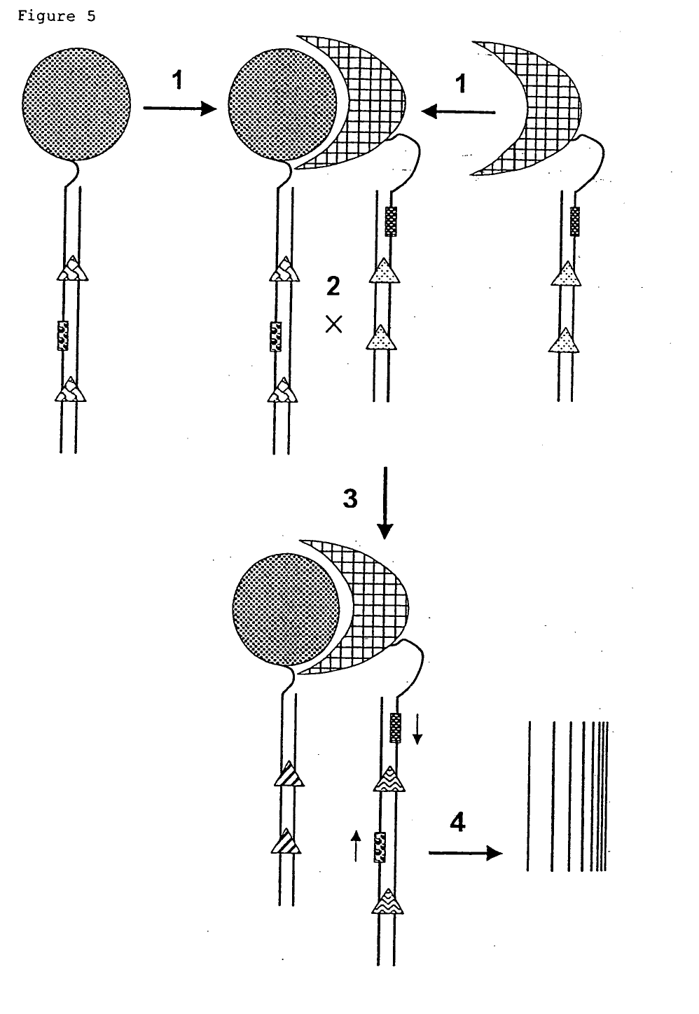

FIG. 5 schematically illustrates an embodiment of the interaction method.

A key to the symbols used in the figures can be found on page 41.

Referring to FIG. 1, the starting reagents are two binding entities (e.g. antibodies, see the key) with attached nucleic acid tags. In this embodiment, one binding entity is labelled with a circular tag, the other with a linear tag. The binding entities are linked through specific binding to the target molecule (Step 1). After allowing the binding entities to bind to the target, recombinase enzyme is added which allows recombination between the AttP site on one nucleic acid tag and the AttB site on the other nucleic acid tag, leading to the generation of (Step 2) a new nucleic acid molecule with a novel detectable sequence (Step 3). In this example the novel sequence comprises primer-binding sites suitable for PCR or a combination of a primer-binding site and promoter that binds an RNA polymerase and as such can be amplified by NASBA or 3SR isothermal amplification technologies (Step 4). In the absence of the target molecule the nucleic acid tags are not brought into close proximity and the recombination and subsequent amplification cannot occur. In a further example both nucleic acid tags are circular and recombination takes place between the AttP and AttB sites, as above. The product of recombination is a larger circle with a novel nucleic acid sequence incorporating sequence from both circular components which can be detected by the methods described above.

FIG. 2 illustrates an exchange of sequence between the nucleic acid tags that generates a sequence with additional functionality, such as an amplifiable segment that can participate in a PCR process. Two target-specific binding entities (e.g. antibodies) with attached nucleic acid tags (that can be linear or circular) are linked through specific binding to the target molecule (Step 1). After allowing the binding entities to bind to the target Cre recombinase is added which allows recombination between the LoxP sites on one nucleic acid tag and the LoxP site on the other nucleic acid tag-(Step 2). Two new nucleic acid molecules are now formed, one of which contains the novel detectable sequence (Step 3). In this example the novel sequence can comprise primer-binding sites suitable for PCR or a combination of a primer-binding site and promoter that binds an RNA polymerase and can be amplified by NASBA or 3SR isothermal amplification technologies (Step 4). In the absence of the target molecule the nucleic acid tags are not brought into close proximity and the recombination and subsequent amplification cannot occur.

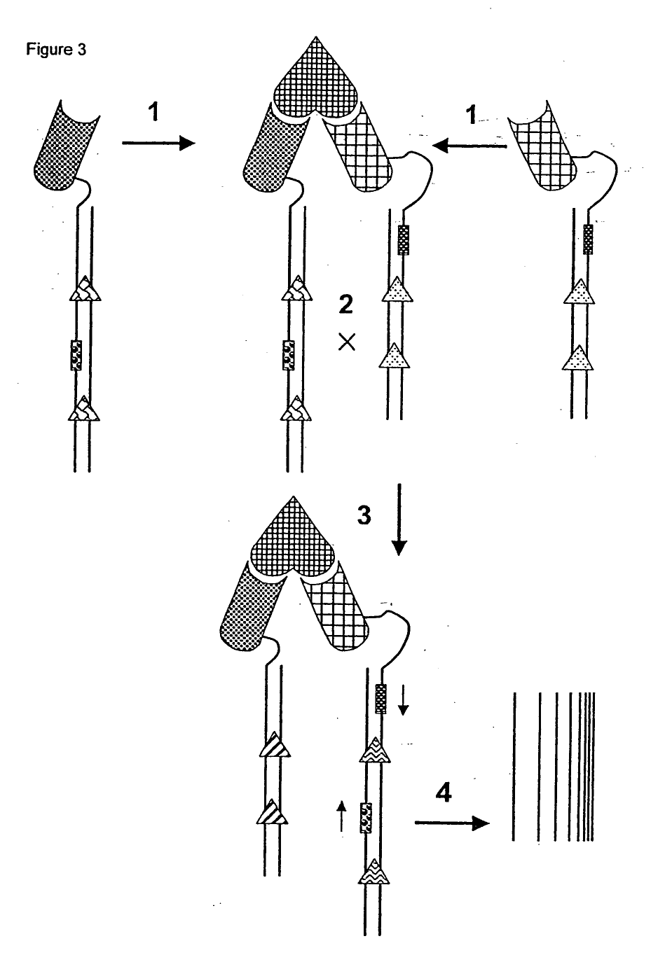

FIG. 3. illustrates an exchange of sequence between the nucleic acid tags that results in a novel detectable sequence. Examples of novel detectable sequences include nucleic acid amplification primer binding sites or RNA polymerase binding sites, i.e. promoters that allow detection of the novel sequence by amplification. The sequences that promote the exchange of sequence are shown as triangles. One system that could be used is the phage lambda-based site-specific recombination system. In this example, the attL and attR recombination sequences recombine in the presence of recombinase to generate attP and attB.

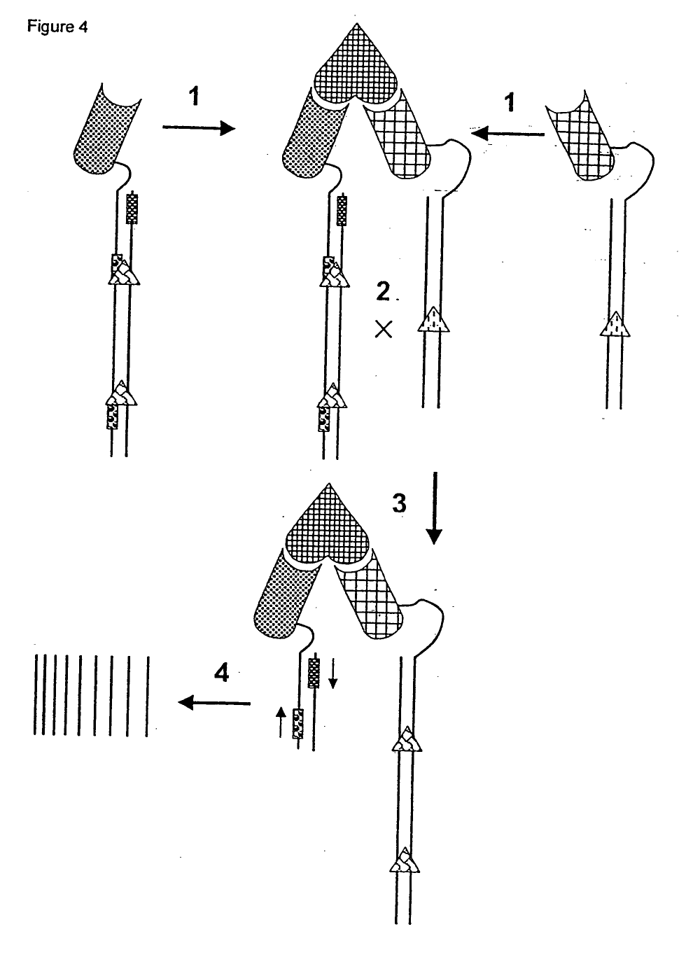

FIG. 4. illustrates an exchange of sequence between nucleic acid tags that exposes a sequence capable of amplification and detection. Two target-specific binding entities are shown with attached nucleic acid tags (that can be linear or circular). The binding entities are linked through specific binding to the target molecule (Step 1). After allowing the binding entities to bind to the target Cre recombinase is added which allows recombination between the LoxP sites on one nucleic acid tag and the LoxP site on the other nucleic acid tag (Step 2). Two new nucleic acid molecules are now formed, one of which contains the novel detectable sequence (Step 3). This novel sequence can contain an amplifiable sequence and can act in conjunction with another site on the same nucleic acid molecule as the basis for PCR, NASBA or 3SR isothermal amplification detection technologies (Step 4). In the absence of the target molecule the nucleic acid tags are not brought into close proximity and the recombination and generation of the novel detectable sequence and subsequent amplification cannot occur.

FIG. 5. illustrates the use of the technology for monitoring of molecular interactions. The molecular entities (interacting molecules) whose interaction is to be studied (the circle and moon in FIG. 5) are directly or indirectly tagged by nucleic acid tags. When the interacting molecules interact the nucleic acid tags are brought into proximity and can interact (see step 1 in the Figure). For example in the recombinase system the tags have recombination-specific recognition sequences (the triangles in the Figure) and in the presence of recombinase an exchange of sequence between the nucleic acid tags (see step 2) results in a novel detectable sequence (the rectangles in the Figure)(see step 3). Examples of novel detectable sequence can be nucleic acid amplification primer binding sites or RNA polymerase binding sites ie. promoters that allow detection of the novel sequence by amplification (step 4). In this example, the attL and attR recombination sequences recombine in the presence of recombinase to generate attP and attB. Such a method can be used to monitor the interaction of molecules that are known to interact, to search for novel interacting molecules or to discover the interaction of known molecules. The method could also be used to observe the inhibition of interaction by the presence of an inhibitor, for example. The latter approach may be used in drug discovery programmes to identify molecules that inhibit the interaction of biologically important molecular entities.

### EXAMPLE 1

### Demonstration of Target-Enhanced Recombination Between DNA Molecules Labeled with Target-Specific Ligands

This experiment illustrates that recombination between two molecules of double stranded DNA is enhanced when the two DNA molecules are-brought into close proximity through binding to a target molecule. In this example the DNA strands are derivatised with biotin which acts as a ligand for the target molecule, streptavidin (each molecule of streptavidin can bind four biotin ligands).

1. The bacteriophage integrase (a phage specific recombinase) signal sequences, attL and attR, necessary for recombination were amplified using the polymerase chain reaction (PCR) from two commercially available plasmids containing these sequences. The PCR primers contained amino terminal groups to allow subsequent chemical derivatisation of the PCR product.

2. The PCR products were agarose gel purified using standard techniques and biotinylated using 0.5 mg of biotinamidohexanoic acid 3-sulfo-N-hydroxysuccinimide ester in PBS containing 1 μg of each PCR product.

3. Biotinylation products were purified by ethanol precipitation and agarose gel purification. J

4. Two reactions were prepared each containing 100 ng of each biotinylated PCR product in 10 μl PBS. One reaction also contained 10¹⁰ molecules of streptavidin.

5. After 2 hours at room temperature to allow the interaction of the biotin ligand with the streptavidin, 0.5 μl 10-fold serial dilutions of each reaction were added to 5 μl recombination reactions containing recombinase and the appropriate buffer.

6. Reactions were incubated at 24° C. for 2 hours.

7. After recombination the reactions were investigated by PCR in order to detect the recombined products. One PCR primer was directed to the sequence 5′ of the attL sequence and one primer was directed to the 3′ of the attR sequence. Any PCR product is indicative of recombination.

8. After PCR the reactions were analysed by agarose gel electrophoresis.

Results

Recombination-specific PCR products were visible in the reactions that had contained the highest concentration of biotinylated DNA. The band observed in the reaction that had contained streptavidin was approximately 5-fold more intense than the reaction that did not contain streptavidin. In the absence of recombinase (ie. no enzyme control) there were no PCR products.

Discussion

This indicates that streptavidin under these conditions promotes the recombination between the double stranded DNA molecules. This is due to the binding of the DNA molecules to streptavidin through the biotin ligands. In this way the DNA molecules are brought into proximity and this enhances the rate of recombination between them.

### EXAMPLE 2

### Demonstration of Molecular Interactions Through Enhanced Recombination Between DNA Molecules Attached to the Interacting Molecules

This experiment illustrates that recombination can be used to monitor molecular interactions. The two reactants are tagged with double stranded DNA. When the molecules interact the two DNA molecules are brought to close proximity and the recombination between them is enhanced. In this example, we have monitored the interaction of streptavidin tagged with one double strand of DNA and biotin tagged with the other double strand of DNA.

1. The bacteriophage integrase (a phage specific recombinase) signal sequences, attL and attR, necessary for recombination were amplified using the polymerase chain reaction (PCR) from two commercially available plasmids containing these sequences. The PCR primers contained amino terminal groups to allow subsequent chemical derivatisation of the PCR product.

2. The PCR products were agarose gel purified using standard techniques and biotinylated using 0.5 mg of biotinamidohexanoic acid 3-sulfo-N-hydroxysuccinimide ester in PBS containing lug of each PCR product.

3. Biotinylation products were purified by ethanol precipitation and agarose gel purification.

4. To form the DNA-tagged streptavidin, streptavidin and biotinylated attL PCR product were mixed in a 1:2 molar ratio and allowed to react for 30 mins.

5. The tagged streptavidin was then immobilized onto a solid phase. In this example the tagged streptavidin was immobilized on maleic anhydride (Reacti-Bind, Pierce) microtiter plate wells following the manufacturers instructions. 10-fold serial dilutions from 10 ng to 10 fg of tagged streptavidin were immobilized. A no-tagged streptavidin control was also included.

6. After immobilization of the tagged streptavidin, the wells were incubated with 10 ng/ml biotinylated attR PCR product in PBS 0.1% (v/v) Tween20. Control wells with immobilized tagged streptavidin were incubated with PBS only.

7. After 60 mins the wells were washed x5 with PBS and 10 μl Cre recombinase (Invitrogen) diluted 1/10 in the supplied reaction buffer was added. Control wells with tagged streptavidin and incubated with attR had no recombinase added.

8. After recombination at 24° C. for 2 hours, the wells were washed x3 with PBS and the recombined products were eluted from the wells by addition of 100 μl distilled water and heating at 100° C. for 10 mins.

9. The eluates were then investigated by PCR in order to detect the recombined products. One PCR primer was directed to the sequence 5′ of the attL sequence and one primer was directed to the 3′ of the attR sequence. Any PCR product was indicative of recombination.

10. After PCR the reactions were analyzed by agarose gel electrophoresis.

Results

Recombination-specific PCR products were visible in all of the eluates that had been derived from wells containing tagged streptavidin. The no-streptavidin control remained negative as did the control wells with tagged streptavidin but no attR incubation. In the absence of recombinase there was no recombination-specific products.

Discussion

This indicates that under these conditions the recombinase assay can be used to monitor molecular interactions at high sensitivity. Further experiments have shown that binding of the tagged streptavidin to the maleic anhydride wells can be replaced by immobilization to magnetic beads or passive adsorption onto plastic surfaces.

EXAMPLE 3

### Demonstration of Target-Specific Recombination Between DNA Molecules Labeled with Target-Specific Ligands

This experiment illustrates that recombination between two molecules of double stranded DNA is enhanced when the two DNA molecules are brought to close proximity through binding to a target molecule. In this example the DNA strands are derivatised with biotin which acts as a ligand for the target molecule, streptavidin (each molecule of streptavidin can bind four biotin ligands). In this example the target is immobilized onto a solid surface before detection.

1. The bacteriophage integrase (a phage-specific recombinase) signal sequences, attL and attR, necessary for recombination were amplified using the polymerase chain reaction (PCR) from two commercially available plasmids containing these sequences. The PCR primers contained amino terminal groups to allow subsequent chemical derivatisation of the PCR product.

2. The PCR products were agarose gel purified using standard techniques and biotinylated using 0.5 mg of biotinamidohexanoic acid 3-sulfo-N-hydroxysuccinimide ester in PBS containing 1 μg of each PCR product.

3. Biotinylation products were purified by ethanol precipitation and agarose gel purification.

4. In this example the streptavidin was immobilized on maleic anhydride (Reacti-Bind, Pierce) microtiter plate wells following the manufacturers instructions. 10-fold serial dilutions from 10 ng to 10 fg of streptavidin were immobilized. A no-streptavidin control was also included.

5. After immobilization of the streptavidin, the wells were incubated with 100 μl PBS 0.1% (v/v) Tween20 containing 10 ng/ml of each attL and attR biotinylated PCR product.

6. After 60 mins the wells were washed x5 with PBS and 10 μl Cre recombinase (Invitrogen) diluted 1/10 in the supplied reaction buffer was added. Control wells, with streptavidin and incubated with attR and attL, had no recombinase added.

7. After recombination at 24° C. for 2 hours-, the wells were washed x3 with PBS and the recombined products were eluted from the wells by addition of 100 μl distilled water and heating at 100° C. for 10 mins.

8. The eluates were then investigated by PCR in order to detect the recombined products. One PCR primer was directed to the sequence 5′ of the attL sequence and one primer was directed to the 3′ of the attR sequence. Any PCR product was indicative of recombination.

9. After PCR the reactions were analysed by agarose gel electrophoresis.

Results

Recombination-specific PCR products were visible in all of the eluates that had been derived from wells containing streptavidin. The no-streptavidin control remained negative as did the control wells which were not incubated with recombinase.

Discussion

This indicates that immobilized target, streptavidin in this example, binds the ligands (biotins) and thus promotes the recombination between the biotin-attached double stranded DNA molecules.

### EXAMPLE 4

### Demonstration of Inhibition of Target-Specific Ligand Binding and the Inhibition of Recombination of the Attached DNA Tags

This experiment illustrates that the recombinase assay can be used to monitor or detect inhibitors that inhibit the binding of target-specific ligands to the target molecule. In this example the ligands (biotin) are derivatised with two different double-stranded DNA molecules that can interact by recombination. The binding of the ligands to streptavidin can be inhibited by the presence of free biotin (the inhibitor).

1. The bacteriophage integrase (a phage specific recombinase) signal sequences, attL and attR, necessary for recombination were amplified using the polymerase chain reaction (PCR) from two commercially available plasmids containing these sequences. The PCR primers contained amino terminal groups to allow subsequent chemical derivatisation of the PCR product.

2. The PCR products were agarose gel purified using standard techniques and biotinylated using 0.5 mg of biotinamidohexanoic acid 3-sulfo-N-hydroxysuccinimide ester in PBS containing 1 μg of each PCR product.

3. Biotinylation products were purified by ethanol precipitation and agarose gel purification.

4. In this example 1 ng of streptavidin was immobilized on-maleic anhydride (Reacti-Bind, Pierce) microtiter plate wells following the manufacturers instructions.

5. After immobilization of the streptavidin, the wells were incubated with 100 μl PBS 0.1% (v/v) Tween20 containing 10-fold serial dilutions of free biotin from 100 ng-0.01 pg and 10 ng/ml of each attL and attR biotinylated PCR products.

6. After 60 mins the wells were washed x5 with PBS and 10 μl Cre recombinase (Invitrogen) diluted 1/10 in the supplied reaction buffer was added.

7. After recombination at 24° C. for 2-hours, the wells were washed x3 with PBS and the recombined products were eluted from the wells by addition of 100 μl distilled water and heating at 100° C. for 10 mins.

8. The eluates were then investigated by PCR in order to detect the recombined products. One PCR primer was directed to the sequence 5′ of the attL sequence and one primer was directed to the 3′ of the attR sequence. Any PCR product was indicative of recombination.

9. After PCR the reactions were analysed by agarose gel electrophoresis.

Results

Recombination-specific PCR products were visible in all of the eluates that had been derived from wells containing 10 pg or less of inhibitor. The signal increased with decreasing quantity of inhibitor. Inhibitor above 10 pg inhibited recombination completely by preventing the ligands from binding to the target streptavidin.

Discussion

This indicates that the recombination assay can be used for detection of inhibitors of ligand binding and that the signal generated is inversely proportional to the degree of inhibition.

1. A method of detecting a target molecule comprising the steps of:
a) contacting a sample with two or more binding entities;
b) allowing the binding entities to bind to the target molecule;
c) allowing interaction between nucleic acid tags attached to the binding entities, wherein the interaction generates at least one tag comprising novel sequence, and wherein the nucleic acid tags are not covalently cross-linked following the interaction;
d) detection of novel sequence in at least one tag generated in step c).

2. A method of detecting interactions between two or more interacting molecules comprising the steps of:
a) incubating the interacting molecules such that they can interact;
b) allowing interaction between nucleic acid tags attached to the interacting molecules, wherein the interaction generates at least one tag comprising novel sequence, and wherein the nucleic acid tags are not covalently cross-linked following the interaction;
c) detection of novel sequence on at least one tag generated in step b).

3. A method according to claim 1 or claim 2 wherein the interaction between nucleic acid tags occurs by recombination.

4. A method of detecting a target molecule comprising the steps of:
a) contacting a sample with two or more binding entities;
b) allowing the binding entities to bind to the target molecule;
c) recombination between nucleic acid tags attached to the binding entities thus generating novel sequence;
d) detection of novel sequence generated by recombination between the nucleic acid tags.

5. A method of monitoring an interaction between interacting molecules comprising the steps of:
a) incubating the interacting molecules together such that they can interact;
b) recombination between nucleic acid tags attached to the interacting molecules thus generating novel sequence;
c) detection of novel sequence generated by recombination between the nucleic acid tags.

6. A method according to claim 4 or claim 5 wherein recombination generates two separate nucleic 30 acid tags, each of which has a novel sequence.

7. A method according to any one of claims 3 to 6 wherein recombination is site-specific and relies upon the use of at least one recombinase enzyme.

8. A method according to claim 7 wherein the recombination is dependent upon attP and attB recognition sequences.

9. A method according to claim 7 wherein the recombination is dependent upon Cre recombinase and LoxP sites.

10. A method according to any one of claims 2 to 6 wherein recombination depends upon the use of at least one transposase enzyme.

11. A method according to claim 10 wherein the recombination depends upon Tn5 transposase that recognizes Mosaic Ends recognition sequences.

12. A method according to any one of claims 7 to 11 wherein the recombinase or transposase is activated after addition to the reaction.

13. A method according to claim 12 wherein the recombinase or transposase is activated by adding an activating buffer to the reaction.

14. A method according to any one of claims 3 to 6 wherein recombination occurs by virtue of homologous recombination between the nucleic acid tags.

15. A method according to any one of the preceding claims wherein the nucleic acid tags are attached directly to the binding entities or interacting molecules.

16. A method according to any one of claims 1 to 14 wherein the nucleic acid tags are attached indirectly to the binding entities or interacting molecules.

17. A method according to any one of the preceding claims wherein the nucleic acid tags comprise of double-stranded DNA or double-stranded RNA.

18. A method according to any one of the preceding claims wherein the nucleic acid tags are linear or circular, or a mixture thereof.

19. A method according to claim 1 or claim 4 wherein the two or more binding entities bind to equivalent sites within identical monomeric units of a multimeric target molecule.

20. A method according to claim 1 or claim 4 wherein the two or more binding entities bind to different regions of the target molecule.

21. A method according to any one of the preceding claims which is carried out in the presence of one or more competitor molecules.

22. A method according to claim 21 wherein the competitor molecules are capable of interacting with at least one of the nucleic acid tags.

23. A method according to claim 22 wherein the competitor molecules can interact with at least one of the nucleic acid tags by recombination.

24. A method according to any one of claims 21 to 23 which is carried out entirely in solution.

25. A method according to any one of the preceding claims wherein the detection of novel sequence is carried out by amplification of at least a part of the novel sequence.

26. A method according to claim 25 wherein amplification is carried out by PCR, NASBA or 3SR.

27. A method according to any one of the preceding claims wherein detection of the novel sequence is carried out in real-time.

28. A method according to claim 27 wherein detection of the novel sequence comprises real-time detection of the product of an amplification reaction using molecular beacons.

29. A method according to any one of the preceeding claims wherein each of the interacting molecules or binding entities are labeled with multiple nucleic acid tags.

30. A method according to claim 29 wherein each interacting molecule or binding entity is labelled with between 2 and 100 nucleic acid tags.

31. A reagent kit comprising two or more binding entities each labelled with nucleic acid tags, characterized in that the nucleic acid tags are capable of interacting to generate at least one tag comprising novel sequence, wherein the nucleic acid tags are not covalently cross-linked following the interaction.

32. A reagent kit for use in monitoring molecular interactions comprising two or more interacting molecules each labelled with nucleic acid tags, characterized in that the nucleic-acid tags are capable of interacting to generate at least one tag comprising novel sequence, wherein the nucleic acid tags are not covalently cross-linked following the interaction.

33. A reagent kit according to claim 31 or claim 32 wherein the nucleic acid tags are capable of interacting by recombination.

34. A reagent kit comprising two or more binding entities each labelled with nucleic acid tags, characterized in that the nucleic acid tags are capable of recombination to generate at least one tag having novel sequence.

35. A reagent kit for use in monitoring molecular interactions comprising two or more interacting molecules each labelled with nucleic acid tags, characterized in that the nucleic acid tags are capable of recombination to generate at least one tag having novel sequence.

36. A reagent kit according to any one of claims 33 to 35 which further comprises a recombinase.

37. A reagent kit according to claim 36 wherein the nucleic acid tags comprise LoxP sites and the recombinase is Cre recombinase.

38. A reagent kit according to claim 36 wherein at least one of the nucleic acid tags contains an attP sequence and at least one nucleic acid tag, other than the tag containing the attP sequence, contains an attB sequence, and the recombinase is capable of catalysing site-specific recombination between attB and attP sequences.

39. A reagent kit according to claim 36 wherein at least one of the nucleic acid tags contains an attL sequence and at least one nucleic acid tag, other than the tag containing the attL sequence, contains an attR sequence, and the recombinase is capable of catalysing site-specific recombination between attL and attR sequences.

40. A reagent kit according to any one of claims 33 to 35 which further comprises a transposase.

41. A reagent kit according to claim 40 wherein the nucleic acid tags comprise Mosaic Ends recognition sequences and the transposase is Tn5 transposase.

42. A reagent labelling kit comprising two or more nucleic acid tags and means for attaching the tags to interacting molecules or to binding entities, characterized in that the kit contains at least one pair of nucleic acid tags which are capable of interacting to generate at least one tag comprising a novel sequence, wherein the two tags are not covalently cross-linked following the interaction.

43. A reagent labelling kit according to claim 42 wherein the nucleic acid tags are capable of interacting by recombination.

44. A reagent labelling kit comprising two or more nucleic acid tags and means for attaching the tags to interacting molecules or binding entities, characterized in that the kit contains at least one pair of nucleic acid tags which are capable of recombination to generate at least one tag having novel sequence.
